(19) United States
(12) Patent Application Publication
(10) Pub. No.: US 2008/0234558 A1
(43) Pub. Date: Sep. 25, 2008

(54) METHODS AND SYSTEMS FOR PERFORMING A CLINICAL ASSESSMENT

(75) Inventors: Vikram S. Kumar, Boston, MA (US); Jonathan Jackson, Boston, MA (US)

Correspondence Address: OCCHIUTI ROHLICEK & TSAO, LLP, 10 FAWCETT STREET, CAMBRIDGE, MA, 02138, US

(73) Assignee: Cogito Health Inc., Cambridge, MA (US)

(21) Appl. No.: 12/050,828

(22) Filed: Mar. 18, 2008

Related U.S. Application Data
(60) Provisional application No. 60/895,868, filed on Mar. 20, 2007.

(51) Int. Cl. A61B 5/00; G06Q 50/00

(52) U.S. Cl. 600/306; 600/300; 705/2; 704/270

(57) ABSTRACT

The invention provides method and systems for performing clinical assessment of a patient that includes determining of a base clinical assessment for the patient by generating information on a clinical rating scale. At least one objective signal is recorded, and each objective signal involves an indicator corresponding to the state of the patient or the state of the patient's environment. Each objective signal is analyzed for generating a corresponding rating on the clinical rating scale. The clinical assessment of the patient may be provided by combining the information from the base clinical assessment with the information generated from analysis of each objective signal. In an embodiment, the clinical assessment may be based exclusively on information generated by analysis of each objective signal. The methods for performing clinical assessment of a patient may also be provided as computer program products having computer readable instructions embodied therein.

### CROSS-REFERENCE RELATED APPLICATION PARAGRAPH

This application claims the benefit of U.S. Provisional Application No. 60/895,868 filed on Mar. 20, 2007. The contents of which is hereby incorporated by reference in its entirety.

### FIELD OF THE DISCLOSURE

This disclosure relates generally to methodology for applying mathematical modeling techniques in the area of medical evaluation, and more specifically to methods and systems for performing a clinical assessment and for improving the reliability of a clinical assessment.

### BACKGROUND

Mathematical modeling techniques are known and include disparate technologies, like Kalman filters, which can work to an end of performing an estimation of a signal by combining data from more than one source.

### SUMMARY

The present disclosure provides methods and systems which allow a user, such as a physician or other clinical care provider, to perform a clinical assessment or to improve the reliability of a clinical assessment through the combination of the assessment with other signals that are recorded from a patient including, but not limited to, voice or motion patterns. In various aspects, the invention allows the physician or clinical care provider to perform a more reliable clinical rating scale.

In an embodiment, the invention provides a method for performing clinical assessment of a patient that includes determining of a base clinical assessment for the patient by generating information on a clinical rating scale. At least one objective signal is recorded, and each objective signal involves an indicator corresponding to the state of the patient or the state of the patient's environment. Each objective signal is analyzed for generating a corresponding rating on the clinical rating scale. The clinical assessment of the patient is provided by combining the information from the base clinical assessment with the information generated from analysis of each objective signal. Alternatively, the clinical assessment may be based exclusively on information generated by analysis of each objective signal.

Each objective signal may be analyzed by relating the signal to the base clinical assessment. Analyzing the objective signals includes application of a mathematical model. The mathematical model may be improved by determining at least one base clinical assessment and recording a corresponding at least one objective signal for a plurality of patients. Each base clinical assessment is obtained at the same time or at nearly the same time as the corresponding objective signal. Each objective signal is then related to a clinical state on the basis of the corresponding base clinical assessment. Alternatively, the mathematical model may be improved by determining a plurality of base clinical assessments and recording a plurality of corresponding objective signals for a specific patient. Each base clinical assessment is determined at the same time or at nearly the same time as the corresponding objective signal. Each objective signal is then related to a clinical state for the specific patient on the basis of its corresponding base clinical assessment. The mathematical model may include a regression approach. Alternatively, the mathematical model may include application of neural networks.

The clinical rating scale may be classified within one of, scales for social health, scales for psychological well being, scales for anxiety, scales for depression, scales for mental status testing, scales for pain measurements, scales for general health status, and scales for quality of life. More specific embodiments of the clinical rating scale may include PHQ-9, visual analog scale for pain, APGAR score for neonatal health, Quality of Life scale, or HAM-D. Without limitation, the invention is used to assess psychiatric diseases (depression, bipolar disease, schizophrenia, anxiety, etc.), endocrine diseases (diabetes, cushings syndrome, thyroid disorders, etc.), cardiac conditions (congestive heart disease, hypertension, peripheral vascular disease, etc.), pain disorders (chronic pain, back pain, etc.), inflammatory diseases (arthritis, inflammatory bowel disease, psoriasis, etc.), neurological conditions (epilepsy, headaches, traumatic brain injury, etc.), and rehabilitation (post cardiac bypass surgery rehabilitation, etc.).

The base clinical assessment may include assessment of the patient by a healthcare provider. The base clinical assessment may alternatively include a self-report performed by the patient.

Objective signals may be recorded periodically, to provide updates to the base clinical assessment. Objective signals may be recorded by a sensor. The objective signal may include galvanic skin conductance or a recorded speech sample from the patient. Where the objective signal is a recorded speech sample, based on the clinical rating generated for the objective signal, the patient may be subjected to an additional clinical assessment on the clinical rating scale. Where the objective signal is a speech sample, the signal may be recorded over a communication device, including a phone, and may be recorded by an Interactive Voice Response (IVR) Server.

The base clinical assessment may also be obtained from a patient over a communication device, including a phone and may be recorded by an IVR Server.

Combining the information generated by the base clinical assessment with information generated by analysis of the objective signal may include application of a mathematical model. The applied mathematical model may include a Kalman filter.

Where the objective signal is a speech sample, it may be analyzed by applying speech analysis techniques to extract voice features. Extraction of voice features may include identification of voiced segments of a speech sample. Voice features are then extracted from voiced segments of the speech sample. Identification of voiced segments in a speech sample includes applying a two-level Hidden Markov Model. The two-level Hidden Markov Model includes use of at least one of autocorrelation, entropy, and residual amplitude structure of the speech samples and may be applied to 30 millisecond speech samples. The identification of voiced segments may be iteratively improved using the Baum-Welch Expectation Maximization technique.

Voice features extracted from a speech sample include Class I voice features and Class II voice features. Class I features include one or more of formant frequency, confidence in formant frequency, spectral entropy, value of largest autocorrelation peak, location of largest autocorrelation peak, number of autocorrelation peaks, energy in frame and time derivative of energy in frame. Class II features include one or more of average length of voiced segment, average length of speaking segment, fraction of time speaking, voicing rate, fraction speaking over, average number of short speaking segments per minute, entropy of speaking lengths and entropy of pause lengths.

The objective signal may be analyzed and correlated to the clinical rating scale by providing inputs from a plurality of models (m) and uniquely corresponding meta models (m′) to a neural network. Information for correlating the objective signal to the clinical rating scale is generated by the neural network on the basis of said inputs. Inputs are provided by the models (m) and meta models (m′) on the basis of voice features extracted from the objective signal. A score on the clinical rating scale is predicted by each model (m). A corresponding confidence rating is provided by each meta model (m′). The confidence rating provided by each meta model (m′) may include a higher rating when the respective model (m) is probabilistically correct, and a lower rating when the respective model (m) is probabilistically incorrect.

In various embodiments of the present invention, the method for performing clinical assessment of a patient may be provided as a computer program product having computer readable instructions embodied therein.

These and other features and advantages of the present disclosure will be apparent to those skilled in the art of statistics driven clinical assessments from a review of the following detailed descriptions along with the accompanying figures.

### BRIEF DESCRIPTION OF THE DRAWINGS

FIG. 1 provides an illustrative flowchart comprehending overall realization of the method of the present disclosure;

FIG. 2 describes a time varying clinical assessment that is used in FIG. 4;

FIG. 3 describes a time varying objective assessment that is used in FIG. 4;

FIG. 4 provides a more detailed illustration of the overall realization of the method of the present disclosure;

FIG. 5 shows an embodiment of the disclosure in which a mathematical model M1 of FIG. 4 is computed based on training the model using the data of the clinical rating scale and signals across many individuals;

FIG. 6 shows an embodiment of the disclosure in which a mathematical model M1 of FIG. 1 is computed based on training the model using the data of the clinical rating scale and signals within a single individual over time;

FIGS. 7, 8 and 9 provides background that motivates an example of a mathematical model M2 that can improve the reliability of a signal (e.g. A in FIG. 4) by combining it with another signal (e.g. B in FIG. 4);

FIG. 10 provides a preferred mode of the present disclosure;

FIG. 11 provides an example of a mathematical model M0 used to extract voice features of FIG. 10; and

FIG. 12 provides an example of the mathematical model M1 used to estimate the mood rating based on the voice features of FIG. 10.

### DETAILED DESCRIPTION

In management of a patient with a particular disease or condition, a physician or other care provider often uses a standard clinical assessment rating scale such as the Hamilton Depression Rating Scale (HDRS/HAM-D) for assessing levels of depression, the APGAR score for assessing neonatal health or the Quality of Life scale for assessing a patient's functional status. A patient may also rate his or her own disease or condition through a scale such as the Patient Health Questionnaire (PHQ-9) for assessing depression or the visual analog scale for pain (which may be used by patients to self-report levels of pain). Such standard clinical assessments are often used in clinical decision making, such as in deciding to change a medication dosage or refer a patient to a different level of medical care. Without providing an exhaustive recitation, clinical rating scales may be classified inter alia as falling within one of, scales for social health, scales for psychological well being, scales for anxiety, scales for depression, scales for mental status testing, scales for pain measurements, scales for general health status, and scales for quality of life.

By way of example, in the case of major depression, the HDRS and PHQ-9 are known to be correlated with the disease or symptom severity. Often after a clinical interview or patient self-assessment, a physician or other care provider will use the numbers from these scales to increase the dose of an anti-depressant, change the class of medications, request the patient to visit a specialist for evaluation, and so on. The numbers generated through these scales form an important part of the medical evaluation. However, there are two limitations to the standard use of clinical rating scales that motivate this disclosure.

Clinical rating scales that have strong subjective components suffer from poor inter-rater reliability. In other words, two physicians or other care providers may rate a patient's mood differently using a clinical rating scale, based on their subjective clinical impressions of the patient. For example, the first field in the standard HAM-D asks the interviewer to score a ‘1’ if he or she thinks the patient indicated sadness, hopelessness, helplessness, or worthlessness only on questioning, or a ‘3’ if the patient communicated these feeling states through non-verbal cues such as facial expression, posture, voice, and tendency to weep. This scoring is subject to the impression of the interviewer, and may differ between interviewers. The higher the tally of such fields, the greater is the severity of depression.

In addition, clinical rating scales are performed at discrete, and often lengthy, time intervals through the course of clinical management of a patient. For example, a patient may be diagnosed with major depression and have an HDRS before initiation of anti-depressant medications. The physician or other care giver may conduct another HDRS at the patient's next visit. This second visit may occur more than four weeks after the initial visit. During the four weeks, the only mood rating that the physician or other care giver may have for the patient would be the initial HDRS performed. This rating scale becomes a poor estimate of the patient's mood rating as time progresses, and the physician or other care giver does not have an effective method or system to improve the reliability of that initial estimate.

The present disclosure addresses these shortcomings by providing both a method to make a clinical assessment more objective by combining it with an objective measurement (e.g. voice analysis), and by providing a method through which more frequent objective measurements may be factored in to update an older clinical assessment. The disclosure also provides a method to improve the overall reliability of a clinical assessment and a method for arriving at a clinical assessment based exclusively on an objective measurement.

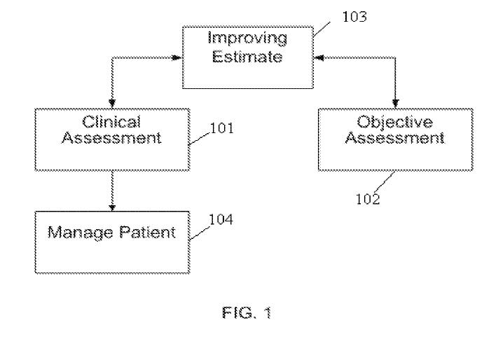

FIG. 1. shows the overall methodology of the disclosure through which an objective assessment 102 may be used to improve an estimate of a clinical assessment 101. This improved or estimated clinical assessment may then be used to manage a patient 104. A clinical assessment is a rating performed on a patient using a standard clinical rating scale such as the PHQ-9. An objective assessment includes signals that may be recorded by a sensor from a patient or his or her environment, such as voice features extracted from a patient's speech.

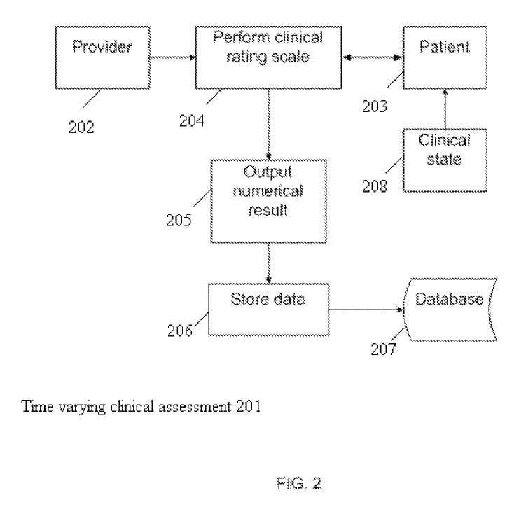

FIG. 2. illustrates how a time varying clinical assessment 201 may be performed. A provider 202 may perform a clinical assessment or clinical rating 204 on a patient 203. A patient may also provide a self report based on the clinical rating scale. The result of the clinical assessment on the rating scale is a numerical score 205 that is stored in a database 207 and is a measure of a patient's clinical state 208. The clinical assessment so determined may be used as a base clinical assessment of the patient.

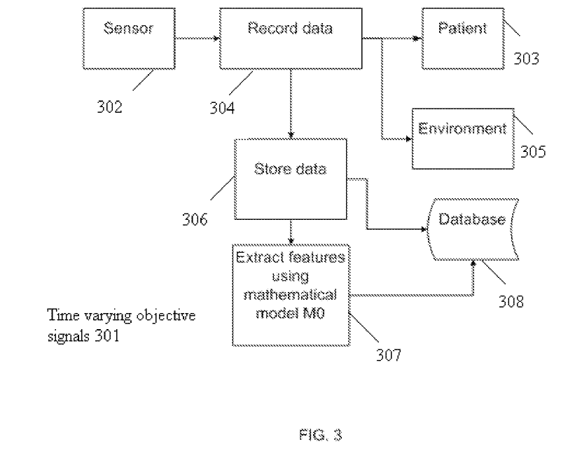

FIG. 3. gives details on how time varying objective signals 301 may be recorded. Objective signals or data from a patient 303 or his or her environment 305 may be recorded, including by way of a sensor 302. A mathematical model (M0) may then be used to extract relevant features from the objective signals or data so recorded. The raw data and extracted features may be recorded in a database 308.

If the recorded objective signal 301 is a speech sample, such sample could provide for extraction of features including the formant frequency, confidence in formant frequency, energy in frame, spectral entropy, value and location of largest autocorrelation peak, number of autocorrelation peaks, time derivative of energy in frame, and average length of voiced segment.

A formant is a resonant frequency and formant frequencies can be found by looking for peaks in the speech signal in the frequency domain. An autocorrelation can be performed to find periodicities within a signal x(t) with mean mx for all lags k=0, 1, 2 . . . N−1.

$$autocorr ( k ) = \frac{{\sum }_{i = 0}^{N - 1} ( {x}_{i} - mx ) ( {x}_{i + k} - mx )}{{\sum }_{i = 0}^{N - 1} {( {x}_{i} - mx )}^{2}}$$

Spectral entropy is a measure of the disorder of a signal in the frequency domain. To arrive at the spectral entropy of a given speech sample, first a probability function of a power spectral density is created based on a magnitude square of the Fourier coefficients. Normalization of the function when done with respect to the total power of Fourier coefficients then yields a probability function used to compute entropy. The mathematical model M0 may use these and other techniques that would be apparent to a person of skill in the art, for extracting relevant features from the recorded speech sample, or from other recorded objective signals.

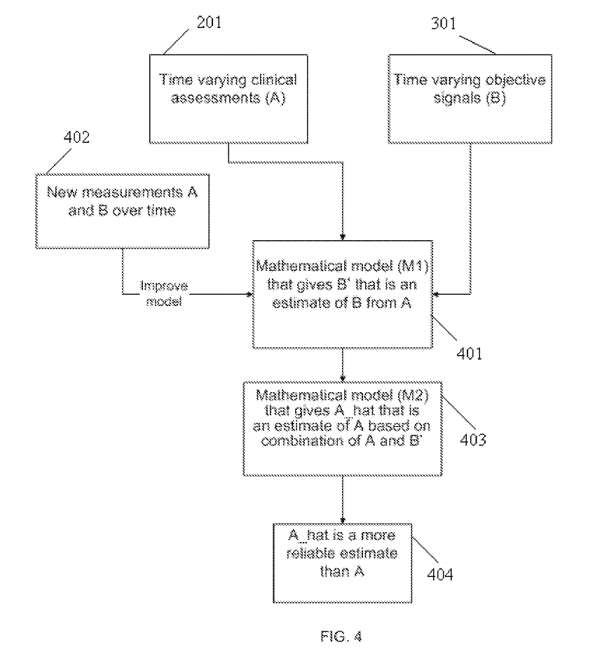

FIG. 4. shows more details on how with the present disclosure, a clinical assessment may be arrived at based on objective signals recorded, or the reliability of a base clinical assessment may be increased using objective signals. An initial or base clinical assessment is performed on a clinical rating scale at a given time as a part of the clinical assessment 201. At the same, or nearly same time, one or more objective signals 301 may be recorded from the patient or the patient's environment. The objective signals recorded may, for example, be the average pitch of a recorded voice sample from the patient or the galvanic skin conductance recorded from the patient. The objective signal or set of signals recorded is then related to the clinical rating scale through data analysis techniques such as a mathematical model M1 401. The method for correlating objective signals with the clinical rating scale may also be applied where the objective signal or set of objective signals is recorded with an interval from the time at which the base clinical assessment is determined. The techniques used for this model M1 may include approaches such as regression or neural networks. An example of an embodiment of M1 is shown in FIG. 12.

Various techniques and mechanisms for achieving the mathematical model M1 would present themselves to a person of skill in the art. In an aspect of the invention, model M1 may be implemented by application of regression. To determine which objective signals are related to a clinical rating scale, a stepwise linear regression can be performed. The goal of said linear regression is to discover the linear combinations of signals which, taken together, would predict the maximum amount of variance in the rating scales and outcomes. This procedure would produce a linear function of the signals that predicts the rating scale. To avoid over fitting and other statistical estimation problems, a cross-validation can be performed including by way of a 5-fold, ‘leave-twenty percent-out’ method, with decision boundaries such that the difference between classification accuracy for the training and test data is minimized.

Implementation of model M1 may also be achieved by way of a neural network. The objective signals may be provided to a Multilayer Perceptron (MLP) or a “blackbox” that creates a network with a single hidden layer and corresponding weights and bias. For neural networks it is proven that there is always a single hidden layer that can approximate a multiple hidden layer. This combination of weighted vectors provides one output that can be correlated with a rating scale. The error of the index and the rating scale indicates how much more training is required for the neural network. A threshold can be set to a 5% change wherein, if an improvement in results is greater then 5% from the previous model, said improved neural network may be used as the modified network. It is understood that the above are only some of the techniques that would present themselves to a person of skill in the art with a view to implement model M1.

The result of the data analysis using M1 is a mathematical equation that relates an objective signal or set of signals at a given time point to the clinical rating scale or disease state at the same time point. By way of example, it may be computed that a model that combines the pitch and energy within a patient's voice at a given time is highly correlated with a patient's PHQ-9 or mood at the same time. As new measurements 402 are performed, the model M1 (401) is improved. Thus the model M1 provides a means to estimate the patient's clinical rating or clinical state at a given time if surrogate measures such as the pitch or galvanic skin response are available in the form of recorded objective signals. Where the objective signal is a recorded speech sample, based on the clinical rating generated for the objective signal, the patient may be subjected to an additional clinical assessment on the clinical rating scale.

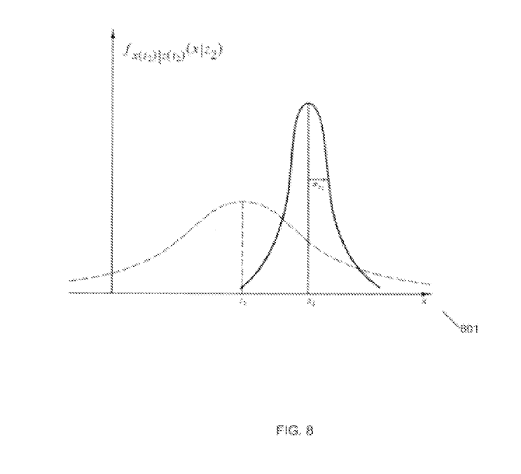

The method then provides an assessment of the patient's clinical state on the clinical rating scale, on the basis of the rating generated by analysis of the objective signal or set of signals. The estimate may be based entirely on the rating generated by analysis of the objective signal or set of signals, or may combine such rating with the initial or base clinical assessments 201 performed on the patient. Another mathematical model M2 (403) may be used to combine the data of 201 and 301 to provide a more reliable estimate of the clinical assessment. Mathematical model M2 achieves this by combining the data 201 and the estimate that M1 makes of the patient's clinical state in terms of the rating scale used to create 201 (e.g. the PHQ-9) based on the data 301 using the relationship M1 derives between 301 and 201. The techniques used for implementing model M2 may include a Kalman filter. FIGS. 7, 8 and 9 give the background behind a Kalman filter that may be used as an M2 to combine the data 201 and 301. The result of M2 would provide an improved or more reliable estimate of the patient's clinical state 404.

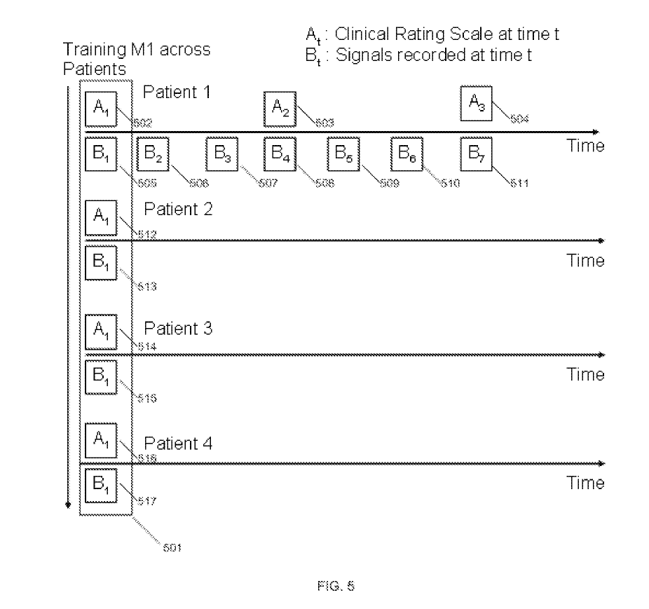

FIG. 5. shows one way in which the model M1 of FIG. 4 may be trained. Many patients have clinical assessments (502, 512, 514, 516) and objective signals recorded (505, 513, 515, 517) that may be used to train M1. Over time, a single patient's objective signals (506, 507, 508, 509, 510, 511) may be related to his or her clinical state using M1. By way of example, a patient may call a computer and leave voice samples over time (506, 507, 508, 509, 510, 511) that are each analyzed and related through M1 to assessments of his or her mood at each time point. An example of M1 is shown in FIG. 12.

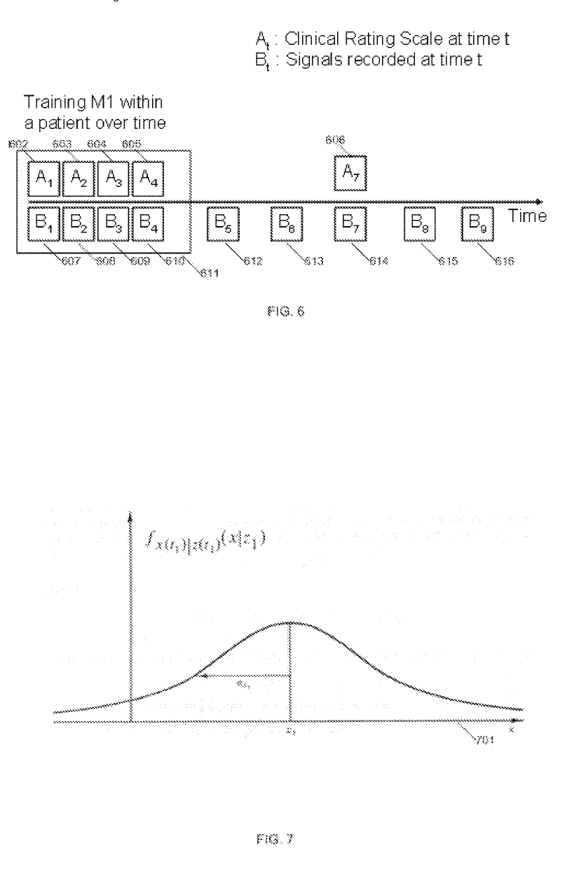

FIG. 6. shows another way in which the model M1 of FIG. 4 may be trained. A single patient has many clinical assessments (602, 603, 604, 605) performed at the same or nearly the same time as objective signals (607, 608, 609, 610) are recorded. M1 is trained on a single patient's data so it becomes a model that shows how that particular patient's objective signals relate to his or her clinical state. The more frequent is the sampling of the clinical assessments and objective signals, the more reliable will be the output by M1.

A scenario in which this type of training could apply is as follows. A patient may use his or her cellular phone to call and perform a PHQ-9 at various time points. With each PHQ-9, the patient may also explicitly leave a voice sample on a computer, or the patient's voice from phone calls completed around the time of the PHQ-9 may be analyzed. The result will be frequent samples of voice and PHQ-9 scores performed around the same times. The data so obtained may be used to train M1. The clinical assessment by way of a rating on the clinical rating scale, and the patient's speech sample can be recorded over phone by an IVR Server.

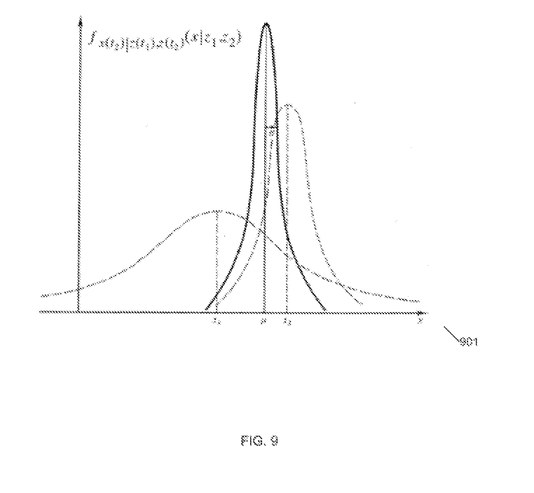

FIGS. 7, 8 and 9 are shown to provide background to how a Kalman filter may be used as the mathematical model M2. FIG. 7. shows a conditional density (701) of an observation (e.g. clinical assessment) based on data z1 (Reference: Stochastic Models, Estimation and Control, Vol. 1, Peter Maybeck, 1979). FIG. 8. shows a conditional density (801) of an observation (e.g. clinical assessment) based on data z2 (Reference: Stochastic Models, Estimation and Control, Vol. 1, Peter Maybeck, 1979). FIG. 9. shows a conditional density (901) of an observation (e.g. clinical assessment) based on the combination of the data z1 and z2 (Reference: Stochastic Models, Estimation and Control, Vol. 1, Peter Maybeck, 1979). The distribution 901 has a lower variance than that of either distribution 701 or 801, demonstrating how the mean of 901 is a more reliable estimate of the observation than either z1 or z2.

The mathematical model discussed in the preceding paragraph is further described by the following relationship:

μ=[σ_{z}_{₂}²/(σ_{z}_{₁}²+σ_{z}_{₂}²)]z₁+[σ_{z}_{₁}²)]z₂1/σ²=(1/σ_{z}_{₁}²)+(1/σ_{z}_{₂}²) (Equation 1

where, μ and σ are the mean and standard deviation of the Gaussian distribution 901 respectively, σ_{z1} and σ_{z2} are the standard deviation of 701 and 801 respectively and z1 and z2 are observations conducted close in time. Equation 1 demonstrates that σ is lower than either σ_{z1} or σ_{z2}.

The final form of the Kalman filter that may be used to implement the mathematical model M2 is:

$$\begin{matrix}x ( {t}_{2} ) = [ {σ}_{{z}_{2}}^{2} / ( {σ}_{{z}_{1}}^{2} + {σ}_{{z}_{2}}^{2} ) ] {z}_{1} + [ {σ}_{{z}_{1}}^{2} / ( {σ}_{{z}_{1}}^{2} + {σ}_{{z}_{2}}^{2} ) ] {z}_{2} \\ = {z}_{1} + [ {σ}_{{z}_{1}}^{2} / ( {σ}_{{z}_{1}}^{2} + {σ}_{{z}_{2}}^{2} ) ] [ {z}_{2} - {z}_{1} ]\end{matrix} \qquad Equation 2$$

where, for example, \hat{x}(t₂) is an estimate of a patient's PHQ-9 score at time t2, z1 is a PHQ-9 result and z2 is a voice feature (that has been converted through mathematical model M1 and is expressed in terms of a PHQ-9 score).

The relationship provided in Equation 3 hereinbelow, relates the estimate of PHQ-9 at time t2 to the estimate of PHQ-9 at time t1 or \hat{x}(t₁).

\hat{x}(t₂)=\hat{x}(t₁)+K(t₂)[z₂ . . . \hat{x}(t₁)]

K(t₂)=σₜ_{₁}²/(σ_{z}_{₁}²+σₜ_{₂}²) Equation 3

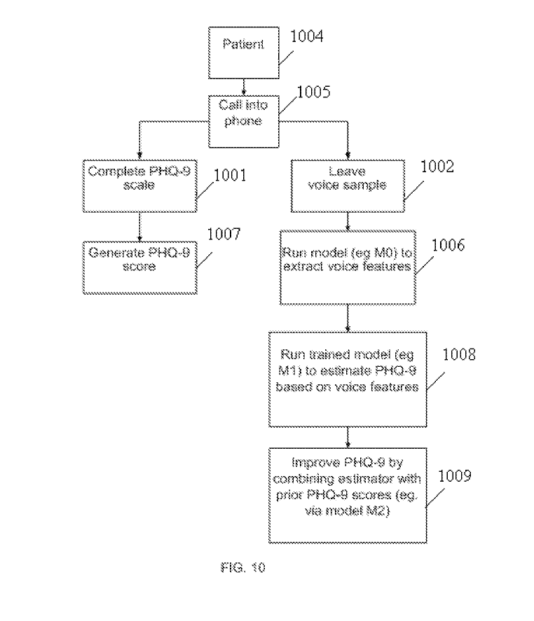

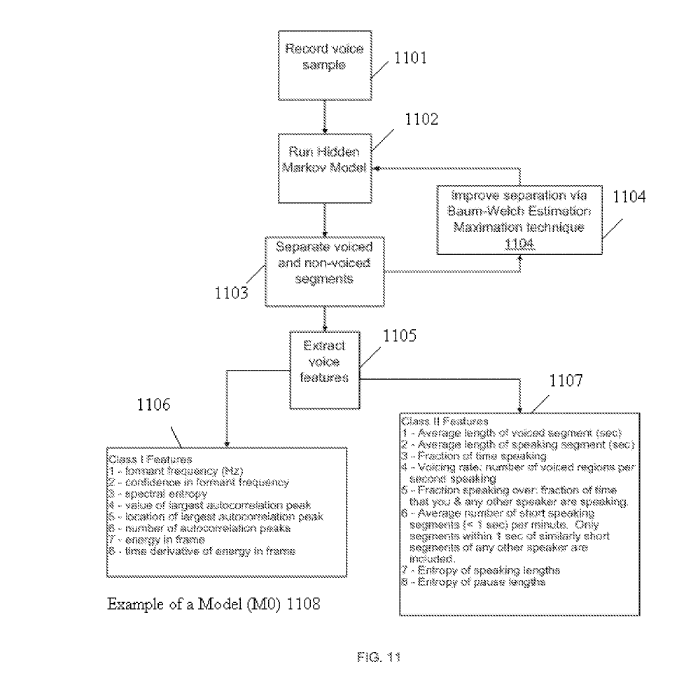

FIG. 10. shows a preferred embodiment that describes the method and system of the disclosure. A patient 1004 calls and performs a PHQ-9 (1001) and leaves a voice sample 1002. A mathematical model M0 (1006) extracts voice features from the voice sample 1002. A trained model M1 estimates PHQ-9 based on the voice features. Finally the clinical assessment is improved in terms of a more reliable PHQ-9 using a model M2 to combine the estimator made in 1008 and other PHQ-9 scores.

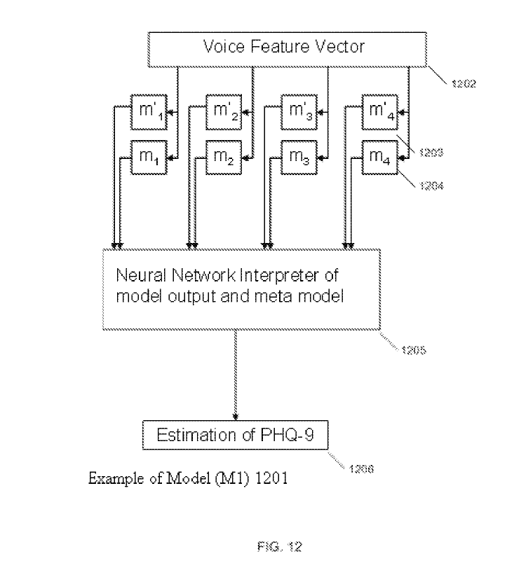

FIG. 11. shows a preferred embodiment that describes how a mathematical model M0 may extract 16 voice features from a voice sample. A voice sample is recorded. Speech analysis techniques such as a Hidden Markov Model (1102) are applied to determine which segments are voiced, and how these segments can be grouped together to constitute a phrase, or a “speaking” segment. This approach is robust to low sampling rates, far-field microphones and ambient noise, all of which can plague real-world situations. Thus, using the raw features described above, a two-level Hidden Markov Model is employed to identify voiced segments (where the vocal folds are vibrating, as in a vowel sound) and group them into speaking regions. This two-level Hidden Markov Model uses at least one of autocorrelation, entropy, and residual amplitude structure of the speech samples. The Hidden Markov Model may apply said techniques to 30 millisecond audio samples. Two states (voice/non voice) are defined over the sequence of 30 ms samples. An initial matrix is fed with random numbers and then the states are guessed. The mathematical model M0 is iteratively improved using the Baum-Welch Expectation Maximization (EM) technique 1104. The mathematical model provides the maximum likelihood estimate thereby allowing the voice sample to be preprocessed by randomly assigning samples to one of the categories and then use the EM algorithm to improve the model. Voice features (1106, 1107) may then be extracted from the preprocessed voice sample using standard techniques including time series analysis (auto regression, auto correlations etc.), information theory (spectral entropy etc.), statistics (averages etc.) and calculus (derivatives etc.).

FIG. 12. shows an embodiment that describes how a mathematical model M1 may use a patient's voice features (1202) to estimate the patient's PHQ-9 score. The voice features may be extracted as described in FIG. 11. Many models m (1203) and their meta models m′ (1204) are trained on a learning dataset as shown in FIG. 5 and FIG. 6. The models m are trained to simply predict the output score such as the PHQ-9. The meta models m′ are trained to output higher scores when their respective model m is likely to be correct and lower scores when m is likely to be wrong. During training, the model m will be trained to give outputs between 0 and 27 according to the PHQ-9 scale while the meta model will be trained to give a confidence rating between 0 and 1.

The outputs of m and m′ are then feed in a Neural Network 1205 that is again trained using as its inputs the outputs from all models and meta models. The neural network uses the m′ 0 to 1 confidence interval as well as the predicted output ms to determine a final output score. Further refinements can be made such that only subsets of the training data are sent to particular models.

The present disclosure uses the surrogate measures both in addition, and also instead of the clinical ratings that are traditionally performed on or by the patient to increase the reliability of the overall clinical assessment. For example, in one embodiment of the present disclosure a patient may perform a PHQ-9 self-report at a first clinic visit and then be asked to call into a phone system and leave a voice sample every other day that an algorithm computes the pitch based upon. As described hereinabove, regular pitch measurements can be combined using a Kalman filter with the original PHQ-9 to provide an ‘updated’ PHQ-9 that gives a more reliable assessment of the patient's depression severity.

References and/or the use of the articles “a” or “an”, unless otherwise specified herein, can be understood to include references to one or more of the noun to which the articles refer. Accordingly, throughout the entirety of the present disclosure, use of the articles “a” or “an”, unless otherwise provided, is for convenience only and is not intended to limit the noun in the singular. Use of the article “the” is also for convenience, and is not intended to limit the modified noun in the singular, and/or otherwise indicate that the disclosed methods and systems are limited to the description/depiction of the modified noun.

Although the methods and systems have been described relative to a specific embodiment thereof, they are not so limited. Obviously many modifications and variations may become apparent in light of the above teachings.

In addition, the method for performing clinical assessment of a patient may be provided as a computer program product having computer readable instructions embodied therein.

Many additional changes in the details, materials, and arrangement of parts, herein described and illustrated, can be made by those skilled in the art. Accordingly, it will be understood that the methods and systems provided herein are not to be limited to the embodiments disclosed herein, can include practices otherwise than specifically described, and are to be interpreted as broadly as allowed under the law.

1. A method for performing clinical assessment of a patient comprising the steps of:
determining a base clinical assessment for the patient comprising generating information based on a clinical rating scale;
recording at least one objective signal, each objective signal comprising an indicator corresponding to the state of said patient or the state of said patient's environment;
analyzing each objective signal for generating a corresponding rating on the clinical rating scale;
providing a clinical assessment of said patient on the basis of information generated by analysis of each objective signal.

2. The method as claimed in claim 1, wherein the step of providing the clinical assessment of said patient comprises combining the information generated by the base clinical assessment with the information generated by analysis of each objective signal.

3. The method as claimed in claim 1, wherein the step of providing the clinical assessment of said patient is based exclusively on information generated by analysis of each objective signal.

4. The method as claimed in claim 1, wherein analysis of each objective signal includes relating said signal to the base clinical assessment.

5. The method as claimed in claim 1, wherein analysis of objective signals comprises application of a mathematical model.

6. The method as claimed in claim 5, wherein the mathematical model is improved by the steps of:
determining at least one base clinical assessment and recording a corresponding at least one objective signal for a plurality of patients, wherein each base clinical assessment is obtained at the same time or at nearly the same time as the corresponding objective signal; and
relating each objective signal to a clinical state on the basis of the corresponding base clinical assessment.

7. The method as claimed in claim 5, wherein the mathematical model is improved by the steps of:
determining a plurality of base clinical assessments and recording a plurality of corresponding objective signals for a specific patient, wherein each base clinical assessment is determined at the same time or at nearly the same time as the corresponding objective signal; and
relating each objective signal to a clinical state for the specific patient on the basis of its corresponding base clinical assessment.

8. The method as claimed in claim 5, wherein said mathematical model comprises application of a regression approach.

9. The method as claimed in claim 5, wherein the mathematical model comprises application of neural networks.

10. The method as claimed in claim 1, wherein the clinical rating scale can be classified as falling within one of, scales for social health, scales for psychological well being, scales for anxiety, scales for depression, scales for mental status testing, scales for pain measurements, scales for general health status, and scales for quality of life.

11. The method as claimed in claim 1, wherein the clinical rating scale comprises one of PHQ-9, visual analog scale for pain, APGAR score for neonatal health, Quality of Life scale, or HAM-D.

12. The method as claimed in claims 1, wherein said clinical rating scale is used to assess the state of any of one, psychiatric diseases including depression, bipolar disease, schizophrenia, and anxiety, endocrine diseases including diabetes, cushings syndrome, and thyroid disorders, cardiac conditions including congestive heart disease, hypertension and peripheral vascular disease, pain disorders including chronic pain and back pain, inflammatory diseases including arthritis, inflammatory bowel disease and psoriasis, neurological conditions including epilepsy, headaches and traumatic brain injury, and rehabilitation including post cardiac bypass surgery rehabilitation.

13. The method as claimed in claim 1, wherein the base clinical assessment comprises assessment by a healthcare provider.

14. The method as claimed in claim 1, wherein the base clinical assessment comprises a self-report performed by the patient.

15. The method as claimed in claim 2, wherein objective signals are recorded periodically, to provide updates to the base clinical assessment.

16. The method as claimed in claim 2, wherein the step of combining the information generated by the base clinical assessment with the information generated by analysis of the objective signals comprises application of a mathematical model.

17. The method as claimed in claim 16, wherein said mathematical model comprises a Kalman filter.

18. The method as claimed in claim 1, wherein each objective signal is recorded by a sensor.

19. The method as claimed in claim 1, wherein the objective signal comprises the galvanic skin conductance recorded from the patient.

20. The method as claimed in claim 1, wherein the objective signal comprises a recorded speech sample from the patient.

21. The method as claimed in claim 20, wherein based on the clinical rating generated for an objective signal, the patient is subjected to an additional clinical assessment on the clinical rating scale.

22. The method as claimed in claim 21, wherein the recorded speech sample is provided over a communication device, including a phone.

23. The method as claimed in claim 1, wherein the base clinical assessment is obtained from the patient over a communication device, including a phone.

24. The method as claimed in claim 23, wherein the base clinical assessment is recorded by an Interactive Voice Response (IVR) Server.

25. The method as claimed in claim 24, wherein the objective signal comprises a speech sample recorded by an IVR.

26. The method as claimed in claim 20, wherein analyzing the objective signal comprises applying speech analysis techniques to extract voice features.

27. The method as claimed in claim 26, wherein extraction of voice features comprises the steps of:
identification of voiced segments of a speech sample; and
extraction of voice features from voiced segments of said speech sample.

28. The method as claimed in claim 27, wherein identification of voiced segments of said speech sample comprises applying a two-level Hidden Markov Model.

29. The method as claimed in claim 28, wherein the two-level Hidden Markov Model uses at least one of autocorrelation, entropy, and residual amplitude structure of speech samples.

30. The method as claimed in claim 29, wherein the two-level Hidden Markov Model uses 30 millisecond speech samples.

31. The method as claimed in claim 30, wherein identification of voiced segments is iteratively improved using the Baum-Welch Expectation Maximization technique.

32. The method as claimed in claim 23, wherein the extracted voice features comprise Class I features and Class II features.

33. The method as claimed in claim 32, wherein said Class I features comprise at least one of formant frequency, confidence in formant frequency, spectral entropy, value of largest autocorrelation peak, location of largest autocorrelation peak, number of autocorrelation peaks, energy in frame and time derivative of energy in frame.

34. The method as claimed in claim 32, wherein said Class II features comprise at least one of average length of voiced segment, average length of speaking segment, fraction of time speaking, voicing rate, fraction speaking over, average number of short speaking segments per minute, entropy of speaking lengths and entropy of pause lengths.

35. The method as claimed in claim 26, wherein the step of analyzing the objective signal and correlating it to the clinical rating scale comprises providing inputs from a plurality of models (m) and uniquely corresponding meta models (m′) to a neural network for generating information correlating said objective signal to the clinical rating scale, wherein said models (m) and meta models (m′) provide said inputs on the basis of voice features extracted from the objective signal.

36. The method as claimed in claim 35, wherein each model (m) predicts a score on the clinical rating scale.

37. The method as claimed in claim 36, wherein each meta model (m′) provides a confidence rating to the neural network.

38. The method as claimed in claim 37, wherein said confidence rating comprises a higher rating when the respective model (m) is probabilistically correct, and a lower rating when the respective model (m) is probabilistically incorrect.

39. A computer program product for performing clinical assessment of a patient comprising a computer readable medium having computer readable program code for:
obtaining a base clinical assessment for the patient comprising information based on a clinical rating scale;
recording at least one objective signal, each objective signal comprising an indicator corresponding to the state of said patient or the state of said patient's environment;
analyzing each objective signal for generating a corresponding rating on the clinical rating scale, said analysis including reference to the base clinical assessment;
providing a clinical assessment of said patient on the basis of information generated by analysis of each objective signal.

40-76. (canceled)
